(19) United States
(12) Patent Application Publication
(10) Pub. No.: US 2005/0015024 A1
(43) Pub. Date: Jan. 20, 2005

(54) Ultrasonic method and device for lypolytic therapy

(76) Inventor: Eilaz Babaev, Minnetonka, MN (US)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 10/916,262

(22) Filed: Aug. 11, 2004

Related U.S. Application Data
(63) Continuation of application No. 10/092,235, filed on Mar. 6, 2002.

(51) Int. Cl. A61N 7/00

(52) U.S. Cl. 601/2

(57) ABSTRACT

The invention relates to an ultrasound transducer for use in therapy or diagnostics. More particularly, it can be used successfully in lypolytic therapy. Said ultrasound transducer comprises different segments, which allows changing curvature radius and consequently focal distance. In this case, depth and volume in treating adipose tissue (lypolytic therapy) is controllable, which means tissue can be treated selectively. Use of the liquid bag between transducer and skin surface allows propagation of ultrasound waves to the target area. After identifying fatty tissue or lypolytic depth, ultrasound transducer must be adjusted for needed focal distance and deliver ultrasound energy for treatment

### FIELD OF INVENTION

This invention relates to ultrasound methodology. More particularly, this invention relates to the use of a variable focal point ultrasonic transducer to lyse adipose or needless tissue by causing an effect which is cavitation- and temperature-based.

### BACKGROUND OF INVENTION

Ultrasonic liposuction, the surgical procedure for removal of fat from storage sites in the body, has grown in popularity. Useful ultrasonic liposuction devices have made it possible to remove fatty tissue with comparative safety. See, for example, U.S. Pat. No. 4,886,491 (Parisi et al.), U.S. Pat. No. 5,823,990 (Henley), U.S. Pat. No. 5,419,761 (Narayanan), and U.S. Pat. No. 6,071,260 to (Halverson). However, those technologies require an invasive open surgical operation and the ultrasonic tip must have direct physical contact with the fat tissue being removed.

Other technologies, such as are disclosed in U.S. Pat. No. 5,143,063 (Fellner), U.S. Pat. No. 6,047,215 (McClure), U.S. Pat. No. 5,209,221 (Riedlinger), U.S. Pat. No. 5,601,526 (Chapelon, et al.), and U.S. Pat. No. 6,113,558 (Rosenschein), are based on the use of focused electromechanical or ultrasound energy for lysing, destroying fat tissue cells in a non-invasive manner. U.S. Pat. No. 5,884,631 (Silberg) and U.S. Pat. No. 6,071,239 (Cribbs), teach injecting a tumescent solution among the fat cells or soft tissue before a sonication process. Furthermore, U.S. Pat. No. 5,624,392 (Oppelt) illustrates the use of focused ultrasound for prostate treatment.

All the above technologies are based on localized heating effect produced at a single focal point by ultrasound waves, and they suffer from the major shortcoming of having an ultrasound transducer with a single, fixed focal point. A common problem often associated with focused ultrasonic transducers is the inability to accurately control the depth and/or the volume of a given treatment or application regimen because of the single, fixed focal point.

In actuality, different patients have varying depths of adipose tissue, and this further varies by the location of the tissue. Accordingly, there is a need for an ultrasound transducer where the focal point can be adjusted. A number of U.S. patents are directed to solving this problem: U.S. Pat. No. 5,735,282 (Hossack John), U.S. Pat. No. 6,071,239 (Cribbs et al.), and U.S. Pat. No. 6,042,556 (Beach et al.) disclose the use of multiple ultrasonic transducer elements, which differ in curvature. Those transducer elements must be located on a non-rigid (i.e., elastic) platform, where changing the arc or radius of curvature allows the focal point to vary. However, use of an elastic platform for multiple transducer elements causes various operational difficulties, including limits on duration of ultrasound application and restrictions that prevent rigid piezo-composite or ceramic ultrasound transducers from being used.

High intensity, focused ultrasound (HIFU) has previously been used successfully to destroy tissue, create hypothermia, melt fatty tissue, and deliver effective therapeutic doses to targeted areas. High intensity, focused ultrasound transducers manufactured by IMASONIC, of Besancon, France, use this principle. However, these have only been used in single focal point applications.

The frequencies of ultrasound waves described in the above mentioned applications are typically in the MHz range and with intensities up to 100% w/cm². However, such procedures have a decided drawback in that the temperature in a focal zone is raised to about 40° C.

### OBJECT OF THE INVENTION

It is an object of the invention to provide an improved method and device for an ultrasound-assisted, non-invasive liposuction and body contouring technique.

It is also an object of this invention to provide a method and device for treating tissue cells using ultrasonic waves.

It is a further object of this invention to provide a method and device for live tissue treatment that provides a changeable, flexible and controllable focal point or depth for treatment.

It is yet a further object of this invention to provide a method and device for live tissue treatment that provides a changeable, controllable volume and weight of treated tissue cells.

These and other objects of the invention will become more apparent from the discussion below.

### SUMMARY OF THE INVENTION

The present invention is directed to making lypolytic therapy practical for treatment depth and weight/volume control as well as adipose tissue removal by using high intensity, focused ultrasound (HIFU) to selectively destroy fat cells non-invasively, i.e., without an invasive or surgical procedure. In a method according to the invention a user can change the focal point of a transducer over a wide range. Consequently, this provides the opportunity to treat fatty or adipose tissue cells at any depth and to any needed volume/weight.

A device of present invention comprises an ultrasound transducer with a segmented construction, much like a bud. This design allows changing the radius of curvature of the transducer and, thereby, its focal point depth, in a very easy, sharp, and quick manner. The simplicity of varying the focal point proves most effective when applied to the adipose tissue at different depths and locations.

### BRIEF DESCRIPTION OF THE DRAWINGS

FIG. 1 is a schematic view of a high intensity, focused ultrasound system for lypolytic therapy with an imaging system;

FIGS. 2A to 2C illustrate different focal distances based on correspondingly different radii of curvature;

FIG. 3 is a schematic, lateral cross-sectional representation of a flexible ultrasound transducer with different focal point distances;

FIG. 4A is a lateral cross-sectional view of a segmented ultrasound transducer with a changeable focal point distance;

FIGS. 4B and 4C are rear and rear oblique views, respectively, of the transducer of FIG. 4A;

FIG. 5A is a lateral cross-sectional view of the segmented ultrasound transducer of FIG. 4A in an “open” position;

FIGS. 5B and 5C are rear and rear oblique views, respectively, of the transducer of FIG. 5A;

FIG. 6 is a schematic, lateral cross-sectional view of bifocal ultrasound transducer;

FIG. 7 is a schematic, lateral cross-sectional view of use of a segmented ultrasound transducer with a liquid bag; and

FIGS. 8A and 8B are schematic, lateral cross-sectional views of systems for changing the focal distance.

### DETAILED DESCRIPTION OF THE INVENTION

The present invention is a method and device which uses ultrasound wave energy for lypolytic therapy with an operational frequency range from about 1 kHz to about 50 MHz. Use of high frequency ultrasound is beneficial to treating tissue based on temperature and cavitation effects. Use of low frequency ultrasound creates mechanical-vibratory lysing, i.e., fragmentation of adipose tissue, cavitation, and temperature effects for treating tissue.

As shown in FIG. 1, the device of the present invention comprises an ultrasound transducer 2, an electrical signal generator 4, a diagnostic or image generator or monitor 6, and a camera or image transducer 8. Due to the curvature of the radiation surface 10 of transducer 2, ultrasound beams 12 are directed to a focal point 14. Camera or image transducer 8, located in the center of ultrasound therapy transducer 2, allows determination and transducer-positioning with respect to location of adipose tissue before treatment. An operator (not shown) controls the therapy by viewing treatment on monitor 6.

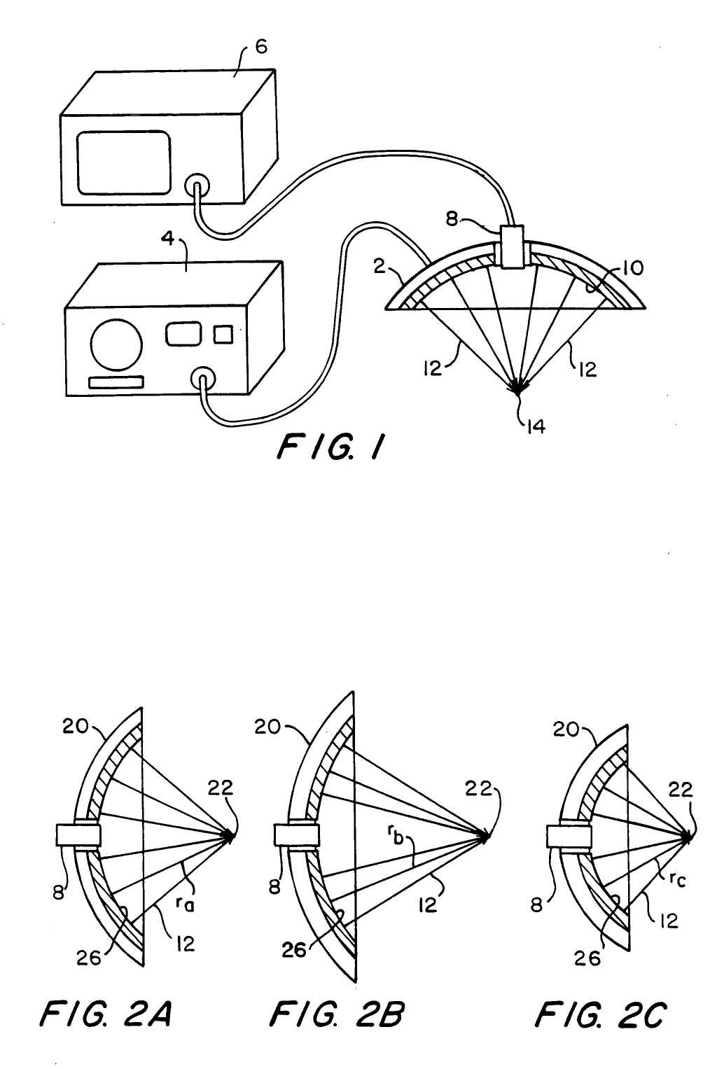

FIGS. 2A to 2C illustrate the basic concept of achieving different focal distances dependent upon a transducer's radius of curvature. In each of FIGS. 2A to 2C, a transducer 20 has a radius of curvature rₐ, r_{b}, r_{c}, respectively, and a focal point 22 for beams 12. The distance of focal point 22 from the surface 26 of transducer 20 in FIGS. 2A to 2C, respectively, is proportional to the respective radii of curvature rₐ, r_{b}, r_{c}. In FIGS. 2A to 2C, r_{c}, <rₐ, <r_{b}.

In adipose tissue treatment, significant therapeutic effect can be achieved by applying focused ultrasound to varying depths and/or locations within a living body. This flexibility, i.e., the ability to change or control treatment depth/volume/area, is characteristic of the device of the present invention.

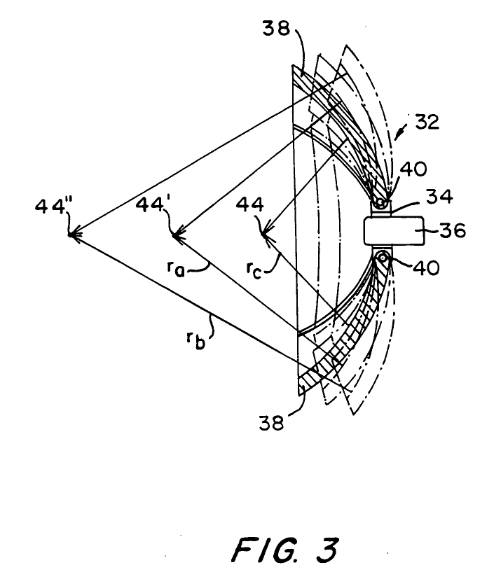

FIG. 3 represents a lateral cross-sectional view of an embodiment of the invention wherein a single transducer can be adjusted to vary the focal point of its transmission. Transducer 32 has a central section 34 that contains a camera or image transducer 36. Transducer segments or sections 38 are each attached to central section 34 at a hinge or knuckle joint 40. The inner, radiating surface 42 of each transducer section 38 having a radius of curvature r_{c} radiates ultrasound radiation that focuses on focal point 44.

In FIG. 3 dotted lines are used to demonstrate alternate positions of sections 38, wherein the respective sections will have longer radii of curvature rₐ, r_{b} and different focal points 44′ and 44″. Here, r_{c}<rₐ<r_{b}.

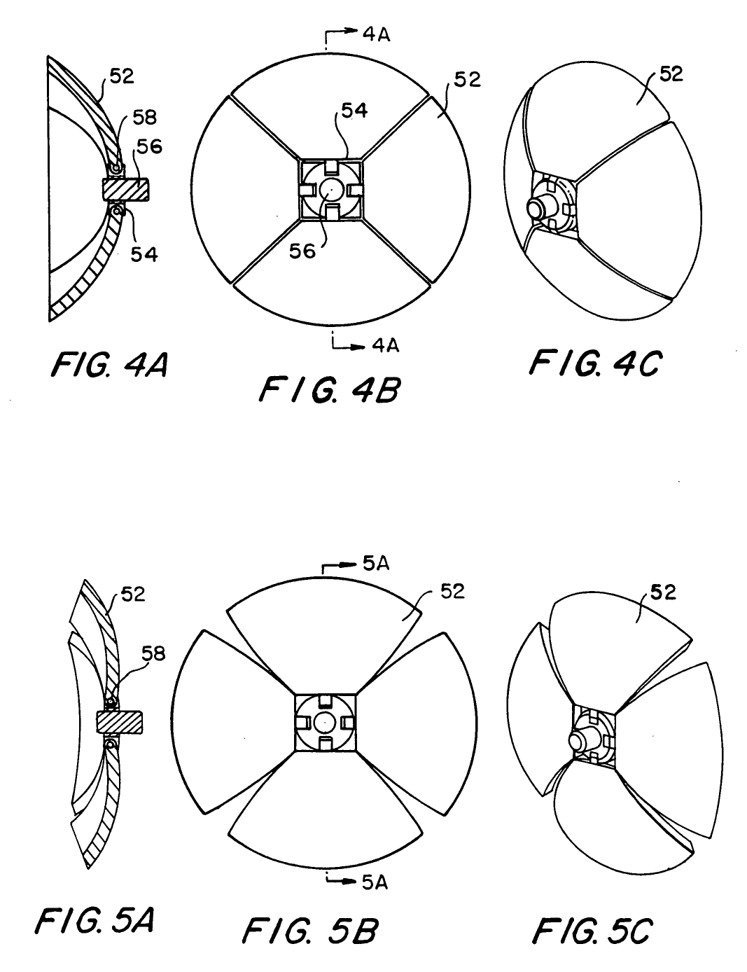

FIGS. 4A to 4C represent an embodiment of the invention wherein four transducer sections 52 are arranged around a central section 54, which contains a camera or image transducer 56. Each section 52 is connected to central section 54 via a knuckle joint or hinge 58.

In FIGS. 5A to 5C transducer sections 52 have been rotated slightly back as compared to the plane of central section 54. This changes the radius of curvature of the transducer and thus the focal point of the ultrasound radiation.

Although the transducer in FIGS. 4A to 5C is shown to have four transducer sections, the number of sections could vary from 2 to 6, or 8, or any practical, preferably even, number. Also, the relative positioning of the transducer sections could be adjusted manually, mechanically, or remotely and/or automatically or electronically through the use of sensors, or servocontrollers or motors. Preferably a control system will have an electronic system whereby the transducer sections can be adjusted quickly, precisely, and uniformly responsive to manual or pre-programmed control.

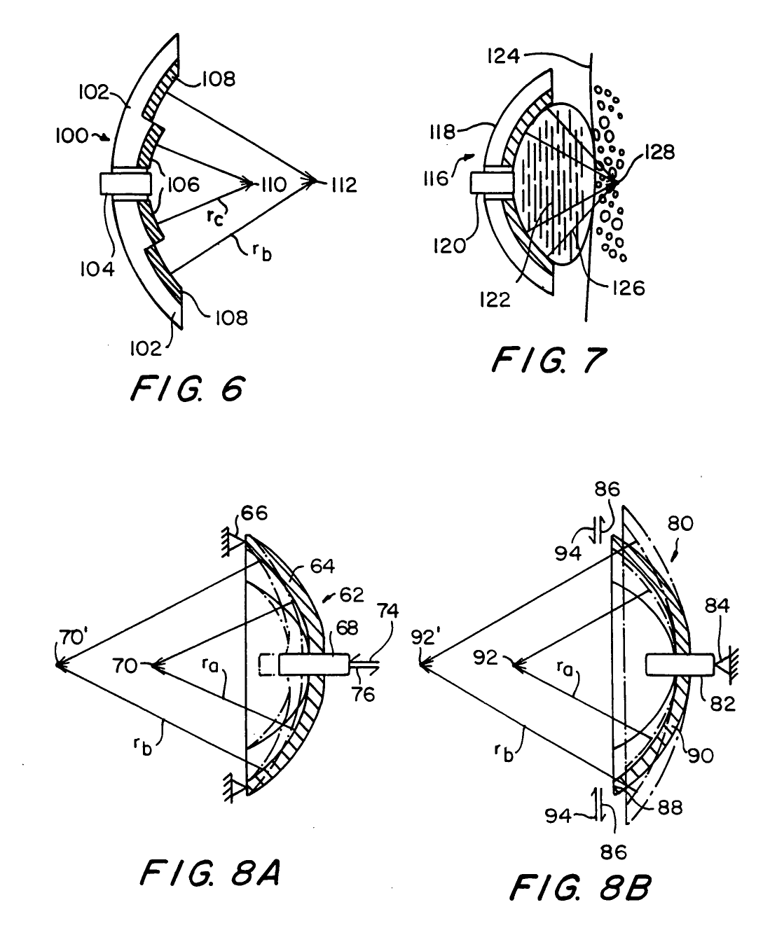

FIG. 6 represents is a lateral cross-sectional slice of a transducer 100 where transducer segments 102 are fixedly attached to a central section 104. The transducer segments 102 have at least two surfaces 106 and 108 having different radii of curvature. However, surface 106 has a radius of curvature rₑ, with a focal point 110, and surface 108 has a radius of curvature r_{c} with a focal point 112.

In FIG. 7, a transducer 116 has transducer segments 118 and a central section 120. A balloon or cushion 122 containing an appropriate liquid, such as sterile water, is positioned between transducer 116 and a working surface 124, such as a patient's skin. Beams of radius 126 focus at focal point or area 128 within adipoise tissue 130.

FIG. 8A is a lateral cross-sectional view of a transducer system 62 where transducer segments 64 are attached to a fixed point or bracket 66. When force is applied to the central section 68, the focal point changes. Here transducer 62 initially has a radius of curvature rₐ and a focal point 70. Then, when force is applied in the direction of arrow 74, the transducer segments 64 move, and the newly positioned segments have a radius of curvature of r_{b} and a focal point 70. Applying force to the direction of arrow 76 reverses the procedure.

Similarly, in FIG. 8B a transducer system 80 has a central section 82 attached to fixed point or bracket 84. When force in the direction of arrow 86 is applied to the outer end 88 of transducer segment 90, the radius of curvature rₐ changes to r_{b} and the focal point changes from 92 to 92′. Force in the direction of arrow 94 reverses the procedure.

The ability to change the focal distances of an ultrasound transducer are critical and highly effective in therapy and diagnostics applications. This flexibility allows one skilled in the art to treat different body parts, at different locations and at different volumes of adipose tissue/fat, with the same transducer in one procedure. An ultrasound transducer 1 can be operated in a continuous mode or in a pulsed mode, either mode having correspondingly different waveforms. Ultrasound transducer segments 8 can be powered (driven) in unison (together, at the same time) or independently (individually, at different times).

To avoid the skin-heating effect and ultrasound-energy damping, transducer 1 must be located on elastic liquid bag/reservoir 5.

1. A ultrasound system for medical ultrasound treatment, comprising:
a power source and
an ultrasound transducer having a curved radiation surface,
wherein the curvature of the curved radiation surface can be adjusted.

2. The system of claim 1, wherein the curved radiation surface focuses ultrasound energy of a focal point.

3. The system of claim 2, wherein the curvature of the curved radiation surface is adjusted to change the focal point.

4. The system of claim 1, wherein the ultrasound transducer is placed in a rigid non-elastic liquid container.

5. The system of claim 1, wherein the ultrasound transducer is placed in a flexible-elastic liquid container.

6. The system of claim 6, wherein the ultrasonic transducer contains 2, 3, 4, or more flexible segments:

7. The system of claim 1, wherein the ultrasound transducer segments are powered separately/individually

8. The system of claim 6, wherein the ultrasound transducers segments are powered.

9. The system of claim 8, wherein the segments move in unison.

10. The system of claim 1, wherein the ultrasound surface contains a central orifice for a camera or image transducer

11. The system of claim 6, wherein the ultrasound transducer segments must be moved for an instant change of focal point distance.

12. The system of claim 1, wherein the ultrasonic transducer is driven with a constant frequency.

13. The system of claim 1, wherein the ultrasound frequency is modulated,

14. The system of claim 1, wherein the ultrasound frequency is pulsed.

15. The system of claim 13, wherein the ultrasonic transducer is driven with a sinusoidal ultrasound wave.

16. The system of claim 13, wherein the ultrasound wave form is rectangular.

17. The system of claim 13, wherein the ultrasound wave form is trapezoidal.

18. The system of claim 13, wherein the ultrasound wave form is triangular.

19. A method for lypolytic therapy comprising the steps of:
(a) providing a system of claim 1;
(b) positioning the ultrasound transducer adjacent to the surface of the skin of a patient; and
(c) moving the ultrasound transducer around the patient's skin to treat adipose tissue beneath the skin.

20. The method of claim 19, wherein the ultrasound transducer is placed on rigid-non-elastic container.

21. The method of claim 19, wherein the ultrasound transducer is placed on flexible-elastic liquid container.

22. The method of claim 19, wherein the ultrasound transducer is driven with constant frequency to treat adipose tissue.

23. The method of claim 19, wherein the ultrasound frequency is modulated.

24. The method of claim 19, wherein the ultrasound frequency is pulsed.

25. The method of claim 23, wherein the ultrasonic transducer is driven with a sinusoidal ultrasound.

26. The method of claim 23, wherein the ultrasonic wave form is rectangular.

27. The method of claim 23, wherein the ultrasound wave form is trapezoidal.

28. The method of claim 23, wherein the ultrasound wave form is triangular.
